(19) United States
(12) Patent Application Publication
(10) Pub. No.: US 2017/0281899 A1
(43) Pub. Date: Oct. 5, 2017

(54) INFANT SLEEP INDUCING DEVICE

(71) Applicant: GEORGE MICHAEL BOREN, EVANSVILLE, IN (US)

(72) Inventor: GEORGE MICHAEL BOREN, EVANSVILLE, IN (US)

(21) Appl. No.: 15/469,258

(22) Filed: Mar. 24, 2017

Related U.S. Application Data
(60) Provisional application No. 62/315,821, filed on Mar. 31, 2016.

(51) Int. Cl. A61M 21/02; A47D 15/00

(52) U.S. Cl. CPC A61M 21/02; A47D 15/00; A61M 2021/0022

(57) ABSTRACT

An infant sleep inducing device that in application, simulates the vibrations that occur in an automobile to soothe a restless infant. The infant sleep inducing device generally comprising a playback device connected to an amplifier, a tactile transducer, a battery source, a housing that defines an inner chamber, and a mounting bracket. A top panel is releasably attached to the housing and includes a central opening. The playback device, amplifier, and battery source are contained within the inner chamber in such a way as to define a recessed portion in the inner chamber. The recessed portion is aligned with the central opening of the top panel. The tactile transducer is received within the recessed portion and a head of the transducer is aligned to project through the central opening of the top panel and is in direct contact with a surface area, such as a surface portion of the infant carrier to transmit physical vibrational impulses to the surface area.

### BACKGROUND OF THE INVENTION

### 1. Field of the Invention

This invention relates generally to a sleep inducer and infant calmer and more particularly to an infant sleep inducing device which is portable and releasably attaches to an infant carrier such as an infant seat for the benefit of applying vibrations to the carrier in order to simulate the vibrations that occur in an automobile to sooth a restless infant resting in the carrier.

### 2. Brief Description of Prior Art

The use of infant rockers is known in the prior art. Such infant rockers devised and utilized for the purpose of pacifying babies are known to consist basically of familiar, expected and obvious structural configurations, notwithstanding the large number of designs encompassed by the crowded prior art. While these devices fulfill their respective, particular objective and requirements, the prior art does not describe a sleep inducing device that releasably attaches to an infant carrier for the benefit of simulating the vibrations that occur in an automobile to soothe a restless infant.

In this respect, the infant sleep inducing device according to the present invention substantially departs from the conceptual concepts and designs of the prior art, and in doing so provides a device developed for the purpose of simulating the vibrations that occur in an automobile to soothe a restless infant.

Therefore, it can be appreciated that there exists a continuing need for a new and improved infant sleep inducing device which can be used for simulating the vibrations that occur in an automobile. In this regard, the present invention substantially fulfills this need.

### SUMMARY OF THE INVENTION

An infant sleep inducing device generally comprising a playback device connected to an amplifier, a tactile transducer, a battery source, a housing that defines an inner chamber, and a mounting bracket. A top panel is releasably attached to the housing and includes a central opening. The playback device, amplifier, and battery source are contained within the inner chamber in such a way as to define a centrally disposed recessed portion within the inner chamber. The recessed portion is aligned with the central opening of the top panel. The tactile transducer is received within the recessed portion and a head of the transducer is aligned to project through the central opening of the top panel, and in application, the head is in direct contact with a surface area, such as a surface portion of the infant carrier. The device transmits physical vibrational impulses principally as a result of the oscillatory movement of the transducer that is in direct contact with the surface area. Thus, the sleep inducing device is particularly useful in imparting a physical vibration to a surface area, such as, an infant carrier.

According to one aspect of the present invention, there is provided an infant sleep inducing device comprising a housing that defines an inner chamber; a playback device located within the inner chamber; an amplifier positioned within the inner chamber, wherein the amplifier is electrically coupled to the playback device; a device for producing vibrations and/or sound coupled to the amplifier; and means for removably affixing the device to an object. Powering means for providing power to the playback device, amplifier and vibration producing device may also be provided. The powering means may be a rechargeable battery located within the inner chamber of the housing. A USB port in the housing that is electrically coupled to the rechargeable battery may be provided for connecting a charging cable to recharge the battery. According to one aspect of the invention, the vibration producing device is a tactile transducer.

According to one preferred embodiment, a top panel that is releasably attached to the housing and includes a central opening therein is provided. A recessed portion in the inner chamber aligns with the central opening. The recessed portion houses the playback device and the amplifier. The tactile transducer is positioned within the recessed portion and a head of the transducer is aligned to project through the central opening for contact with a surface area.

Accordingly to an alternative embodiment, the tactile transducer is in direct contact with an inner wall of the housing such that, when activated, the transducer transmits vibrations to the housing. In this embodiment, the means for removably affixing may include a mounting plate having a first surface configured to be affixed to an object and a second surface configured to be removably affixed to the housing such that vibrations transmitted from the transducer to the housing are further transmitted to the mounting plate and the object.

According to a further aspect of the invention, the means for removably affixing comprises a mounting plate having a first surface configured to be affixed to an object and a second surface configured to be removably affixed to the housing. The mounting plate includes a projection on the second surface and the housing includes a recess on a surface thereof having dimensions corresponding to the dimensions of the projection to removably engage the projection.

According to another aspect of the invention, the playback device comprises a printed circuit board having a central processing unit and memory to permit playback of multiple different sounds and vibrations. The printed circuit board may further include a Bluetooth module for permitting wireless streaming of sounds and vibrations from an external source.

### BRIEF DESCRIPTION OF THE DRAWINGS

The present invention can best be understood in connection with the accompanying drawings. It is noted that the invention is not limited to the precise embodiments shown in the drawings, in which:

FIG. 1 is a cross-sectional view of an infant sleep inducing device according to a first preferred embodiment of the present invention.

FIG. 2A is a front perspective view of an infant sleep inducing device according to an alternative second preferred embodiment of the present invention

FIG. 2B is a rear perspective view of the infant sleep inducing device shown in FIG. 2A and the associated mounting bracket.

FIG. 3 is a front elevational view of the sleep inducing device shown in FIGS. 2A-2B.

FIG. 4 is a side elevation view of the sleep inducing device shown in FIGS. 2A-3.

FIG. 5 is a bottom view of the sleep inducing device shown in FIGS. 2A-4.

FIG. 6 is a top view of the sleep inducing device shown in FIGS. 2A-5.

FIG. 7 is a cross-sectional view of the sleep inducing device taken along the line 7-7 in FIG. 4.

FIG. 8 is a cross-sectional view of the sleep inducing device taken along the line 8-8 in FIG. 5.

FIG. 9 is a top view of the playback device/printed circuit board of the sleep inducing device according to one preferred embodiment of the present invention.

FIG. 10 is a bottom plan view of the playback device/printed circuit board of the sleep inducing device shown in FIG. 9.

### DESCRIPTION OF THE PREFERRED EMBODIMENT

For purposes of promoting and understanding of the principles of the invention, reference will now be made to the embodiments illustrated in the drawings and specific language will be used to describe the same. It will nevertheless be understood that no limitation of the scope of the invention is thereby intended. The invention includes any alterations and further modifications in the illustrated devices and described methods and further applications of the principles of the invention that would normally occur to one skilled in the art to which the invention relates.

The present invention is directed to an infant sleep inducing device 10 that in application, simulates the vibrations that occur in an automobile to soothe a restless infant. More particularly, the present invention releasably attaches to a surface of a baby carrier so as to produce vibrations directly through the surface it is in contact with. In the broadest context, the infant sleep inducing device of the present invention consists of components configured and correlated with respect to each other so as to attain the desired objective.

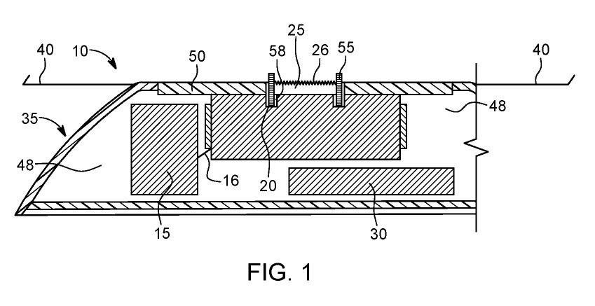

According to the embodiment shown in FIG. 1, the infant sleep inducing device 10 generally comprising a playback device 15 connected 16 to an amplifier 20, a tactile transducer 25, a battery source 30, a housing 35 and a mounting bracket 40. The housing 35 is typically of a rigid material and defines an inner chamber 48. A top panel 50 is releasably attached to the housing 35 and includes a central opening 55 aligned in axially space relation to the approximate center of the chamber 48. The top panel 50 protects the components within the chamber 48, and as will be understood, maintains the transducer 25 within a defined recessed portion 58 in the chamber 48.

The playback device 15, amplifier 20, and battery source 30 are contained within the inner chamber 48 so that the recessed portion 58 is defined near the approximate center of the inner chamber 48 and in application is aligned with the central opening 55 of the top panel 50. The tactile transducer 25 is received within the recessed portion 58 and a head 26 of transducer 25 is aligned to project through the central opening 55. In this relation, the enclosure offers a surface area, such as a surface portion of the infant carrier (not shown) the ability to transmit physical vibrational impulses principally as a result of the oscillatory movement of the transducer 25 that as will be understood, is in direct contact with the surface area. Thus, the enclosure is particularly useful in imparting a physical vibration to a surface area, such as, an infant carrier.

In application, the device 10 is attached to a surface area, such as an infant carrier, using the mounting bracket 40 such that the head 26 of the transducer 25 is in direct contact with the surface area. The sound or audio frequencies transmitted by the amplifier 20 will set up vibrational impulses in the transducer 25. As the transducer 25 is appropriately connected to the amplifier 20, the amplified signal is supplied to the tactile transducer 25 so as to produce the vibrations of the air within the chamber 48. The comfortable coenesthetic acoustic vibrations generated by the tactile transducer 25 are applied to the surface area, namely, the infant carrier as described above, causing the infant carrier to vibrate similar to the vibrations that occur in an automobile to soothe the restless infant resting in the carrier.

As should be apparent, the device 10 in which the tactile transducer 25 is received is portable and releasably attaches to the infant carrier. As such, the device 10 can be removed by releasing the mounting bracket 40 and reattached as described.

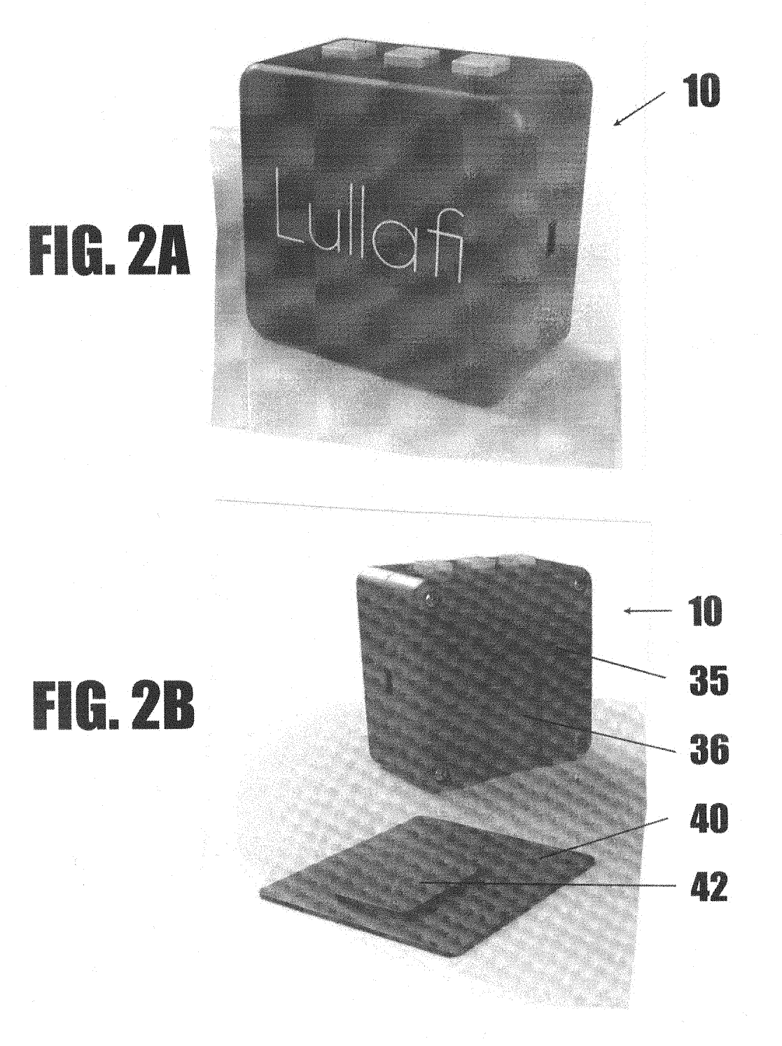

As shown in FIG. 2A and FIG. 2B, the infant sleep inducing device 10 may be removably affixed to an object such as an infant car seat, crib or stroller (not shown) by a mounting bracket 40. The mounting bracket 40 may be affixed to the object in any of a variety of known methods including, but not limited to adhesives. The mounting bracket 40 may be permanently or temporarily affixed to the object depending on the desires of the user. The mounting bracket 40 further includes a projection 42 which engages a recess 36 formed in a rear surface of the housing 35 of the infant carrier device 10 for removably attaching the infant sleep inducing device 10 to the object. The shape and configurations of the projection 42 and recess 36 may vary in any manner necessary to temporarily affix the device 10 to the object, including, but not limited to snap-fit and slidable engagements.

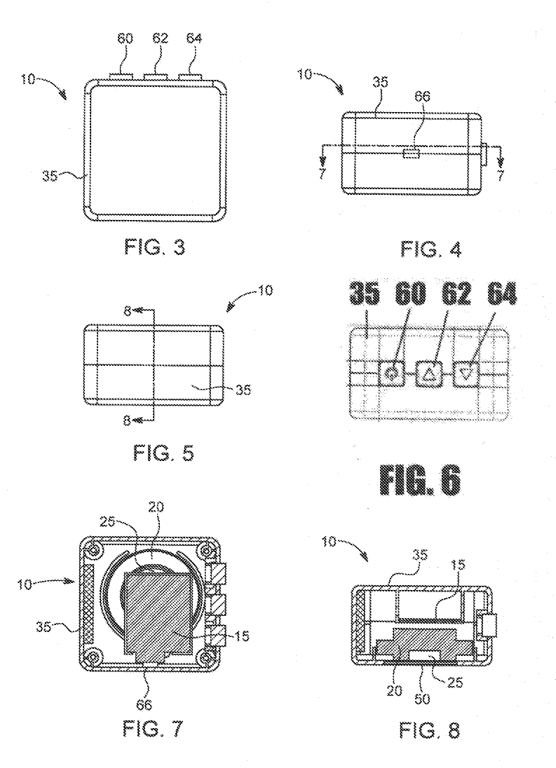

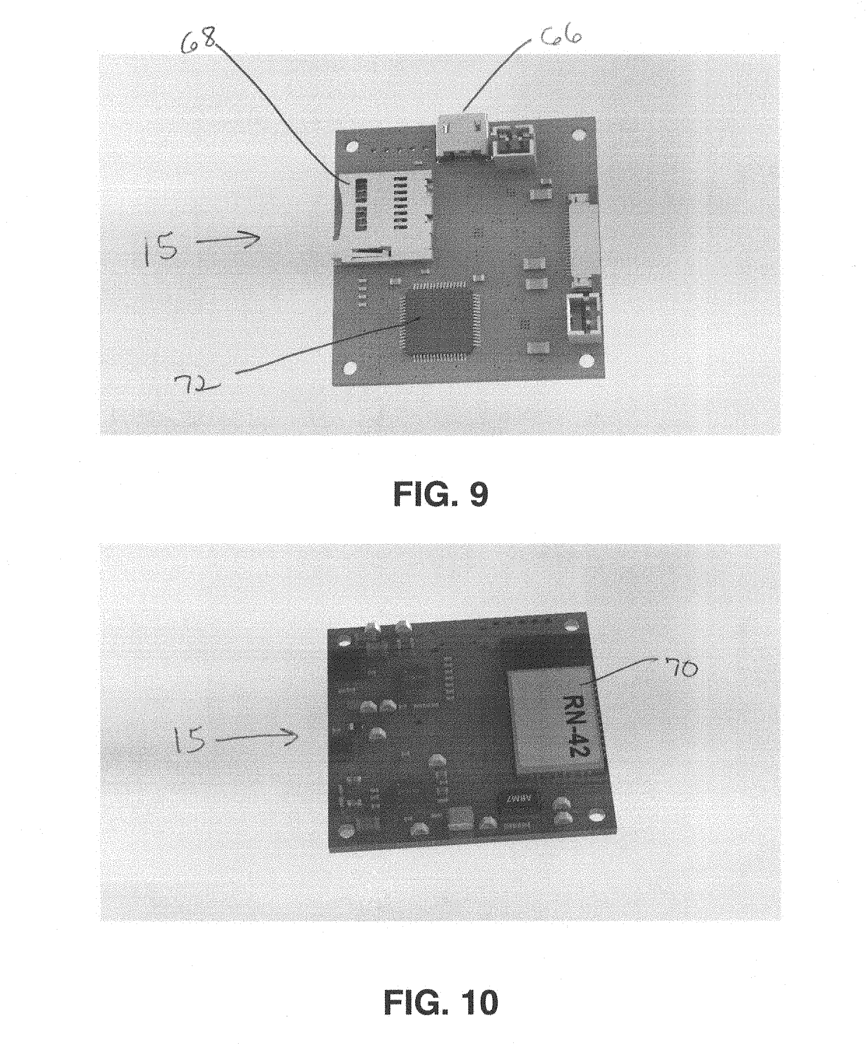

The playback device 15 according to the alternative embodiment shown in FIG. 2A-FIG. 10 is a printed circuit board having a variety of components positioned thereon to effect the production of vibrations and/or sound. Buttons for power 60, increase in volume/vibration frequency 62 and decrease in volume/vibration frequency 64 may be positioned on the upper surface of the case 35, and are electrically connected to the printed circuit board 15 to effect the desired result. A USB port 66 may also be provided in the side of the case 35 and is also electrically coupled to the printed circuit board. The USB port 66 may be used for a variety of tasks such as charging of the internal rechargeable battery that is removably housed in the battery housing 68, or uploading new sounds/vibrations to the unit, for firmware upgrades, or connection to an external source of sound/vibrations or music. Similarly the printed circuit board may be provided with a Bluetooth module 70 that allows users to live-stream any sound, vibration or music, such as classic lullabies, ambient sounds or music, from their Bluetooth compatible device. A central processing unit (CPU) 72 is also located on the PCB 15 to control the various functions of the device. The device's onboard memory contains a plurality of built-in sounds and track programmed with delta sleep patterns that both induce sleep and also train the brain to recognize such sounds as a cue to sleep.

Although the above description contains many specificities, these should not be construed as limiting the scope of the invention but as merely providing illustrations of some of the presently preferred embodiments of this invention. As such, it is to be understood that the present invention is not limited to the embodiments described above, but encompasses any and all embodiments within the scope of the claims.

It would be obvious to those skilled in the art that modifications may be made to the embodiments described above without departing from the scope of the present invention. Thus the scope of the invention should be determined by the appended claims in the formal application and their legal equivalents, rather than by the examples given.

I claim:

1. An infant sleep inducing device comprising:
a housing that defines an inner chamber;
a playback device located within said inner chamber;
an amplifier positioned within the inner chamber, said amplifier electrically coupled to the playback device;
a device for producing vibrations and/or sound coupled to said amplifier; and
means for removably affixing the device to an object.

2. The infant sleep inducing device of claim 1 further comprising powering means for providing power to the playback device, amplifier and vibration producing device.

3. The infant sleep inducing device of claim 2 wherein the powering means is a rechargeable battery located within the inner chamber.

4. The infant sleep inducing device of claim 3 further comprising a USB port in the housing that is electrically coupled to the rechargeable battery for connecting a charging cable to recharge the battery.

5. The infant sleep inducing device of claim 1 wherein the vibration producing device is a tactile transducer.

6. The infant sleep inducing device of claim 5 further comprising:
a top panel that is releasably attached to said housing, said top panel including a central opening therein;
a recessed portion in said inner chamber that aligns with said central opening, said recessed portion housing said playback device and said amplifier; and
said tactile transducer positioned within recessed portion and a head of said transducer is aligned to project through the central opening for contact with a surface area.

7. The infant sleep inducing device of claim 5 wherein the tactile transducer is in direct contact with an inner wall of the housing such that, when activated, the transducer transmits vibrations to the housing.

8. The infant sleep inducing device of claim 7 wherein the means for removably affixing comprises a mounting plate having a first surface configured to be affixed to an object and a second surface configured to be removably affixed to the housing such that vibrations transmitted from the transducer to the housing are further transmitted to the mounting plate and the object.

9. The infant sleep inducing device of claim 1 wherein the means for removably affixing comprises a mounting plate having a first surface configured to be affixed to an object and a second surface configured to be removably affixed to the housing.

10. The infant sleep inducing device of claim 9 wherein the mounting plate includes a projection on the second surface and the housing includes a recess on a surface thereof having dimensions corresponding to the dimensions of the projection to removably engage the projection.

11. The infant sleep inducing device of claim 1 wherein the playback device comprises a printed circuit board having a central processing unit and memory to permit playback of multiple different sounds and vibrations.

12. The infant sleep inducing device of claim 11 wherein the printed circuit board further includes a Bluetooth module for permitting wireless streaming of sounds and vibrations from an external source.
